SEQUENCE LISTING (12 sequences)

SEQ ID NO: 1 - DNA, 423 bp, synthetic construct
  source 1..423: mol_type = other DNA
atgtggctgcagaacctgctgttcctgggcatcgtggtgtacagcctgagcgcccccacaaggtctccaatcacagtgaccagaccctggaagcacgtggaggccatcaaggaggccctgaatctgctggacgacatgcccgtgaccctgaacgaagaggtggaggtggtgtccaacgagttcagcttcaagaagctgacctgtgtgcagaccaggctgaagatcttcgagcagggcctgaggggcaacttcaccaagctgaagggagccctgaatatgaccgccagctactaccagacctactgcccccccacccctgagacagattgcgagacccaggtgaccacctacgccgacttcatcgatagcctcaagacctttctgacagacatccccttcgagtgtaagaagccaggccagaag

SEQ ID NO: 2 - DNA, 762 bp, synthetic construct
  source 1..762: mol_type = other DNA
atgaatttacaaccaattttctggattggactgatcagttcagtttgctgtgtgtttgctctagcttctcaggtacaactgcagcagtctggacctgaactgaagaagcctggagagacagtcaagatctcctgcaaggcctctgggtatcctttcacaaactatggaatgaactgggtgaagcaggctccaggacagggtttaaagtggatgggctggattaacacctccactggagagtcaacatttgctgatgacttcaagggacggtttgacttctctttggaaacctctgccaacactgcctatttgcagatcaacaacctcaaaagtgaagacatggctacatatttctgtgcaagatgggaggtttaccacggctacgttccttactggggccaagggaccacggtcaccgtttcctctggcggtggcggttctggtggcggtggctccggcggtggcggttctgacatccagctgacccagtctcacaaattcctgtccacttcagtaggagacagggtcagcatcacctgcaaggccagtcaggatgtgtataatgctgttgcctggtatcaacagaaaccaggacaatctcctaaacttctgatttactcggcatcctcccggtacactggagtcccttctcgcttcactggcagtggctctgggccggatttcactttcaccatcagcagtgtgcaggctgaagacctggcagtttatttctgtcagcaacattttcgtactccattcacg

SEQ ID NO: 3 - DNA, 69 bp, synthetic construct
  source 1..69: mol_type = other DNA
ttatgggtcatcctgctgagtgcttttgccggattgttgctgttaatgctgctcattttagcactgtgg

SEQ ID NO: 4 - DNA, 45 bp, synthetic construct
  source 1..45: mol_type = other DNA
aagatcggattcttcaagagacctctgaagaagaagatggagaag

SEQ ID NO: 5 - DNA, 252 bp, synthetic construct
  source 1..252: mol_type = other DNA
aagatccatagactgcagagagagaagaagtacaacctggaggtgcctctggtgagcgagcagggcaagaaggccaacagcttccagcaggtgaggagcgacggcgtgtacgaggaggtgaccgccaccgccagccagaccacccctaaggaggcccctgacggccctaggagcagcgtgggcgactgtggccctgagcagcctgagcctctgcctcctagcgacagcaccggcgcccagaccagccagagc

SEQ ID NO: 6 - DNA, 717 bp, synthetic construct
  source 1..717: mol_type = other DNA
gtgagtaagggcgaggagctgtttaccggcgtggtgcccatcctggtggagctggacggcgacgtgaacggccacaagttcagcgtgagcggcgagggggagggcgacgccacctacggcaagctgaccctgaagttcatttgcacaaccggcaagctgcccgtgccatggcctaccctggtgaccaccctgacatacggcgtccagtgttttagcaggtatcccgaccacatgaagcagcacgacttctttaagagcgccatgcccgagggctacgtgcaggagaggaccatcttcttcaaggacgacggtaactacaagaccagagccgaggtgaagttcgagggcgacaccctggtgaaccggatcgagctgaagggcatcgacttcaaggaggacggtaacatcctgggccacaagctggagtataactataatagccacaacgtgtacatcatggccgacaagcagaagaacggcattaaggtgaacttcaagatcagacataacatcgaggacggatctgtgcagctggccgaccactaccagcagaacacccctatcggagatggaccagtgctgctgcctgataatcactatctgagcacacagtctgccctgagcaaagatcctaatgaaaagagagatcacatggtgctgctggagtttgtgacagctgctggaataacactgggcatggatgagctgtataagtaa

SEQ ID NO: 7 - DNA, 57 bp, synthetic construct
  source 1..57: mol_type = other DNA
ggcggtggttccggcggtggatctggtggaggaactggaggaggttcaggaggtggt

SEQ ID NO: 8 - DNA, 66 bp, synthetic construct
  source 1..66: mol_type = other DNA
ggaagcggagctactaacttcagcctgctgaagcaggctggagacgtggaggagaaccctggacct

SEQ ID NO: 9 - DNA, 432 bp, synthetic construct
  source 1..432: mol_type = other DNA
atgtggctgcagtccctgctgctgctggggacagtggcctgcagcatcagcgcccccgccaggagccccagccccagcacacagccctgggagcacgtgaacgccatccaggaggcccggcggctgctgaacctgagcagggataccgccgccgagatgaacgagaccgtggaggtcatcagcgagatgtttgacctgcaggagcccacatgcctgcagacccggctggagctgtacaagcagggcctgagggggtccttgacaaagctgaagggccccctgacaatgatggccagccactacaagcagcactgcccccccacccccgagacctcctgcgccacccagatcatcaccttcgagagcttcaaggagaacctgaaggatttcctgctcgtgatccccttcgactgctgggagcccgtgcaggag

SEQ ID NO: 10 - DNA, 69 bp, synthetic construct
  source 1..69: mol_type = other DNA
ctgtgggtcatcctgctgagcgccttcgccgggctgctgctgctgatgctgctgatcctggccctgtgg

SEQ ID NO: 11 - DNA, 45 bp, synthetic construct
  source 1..45: mol_type = other DNA
aagatcggcttctttaagcggcccctgaagaagaagatggagaag

SEQ ID NO: 12 - DNA, 183 bp, synthetic construct
  source 1..183: mol_type = other DNA
cggaaggagctgaagaggaagaagaagtgggacctggagatcagcctggatagcggccacgagaagaaggtcatctccagcctgcaggaggataggcacctggaggaggagctgaagtgccaggagcagaaggaggagcagctgcaggagggggtgcaccggaaggagccccagggcgccaca

(19) United States
(12) Patent Application Publication
(10) Pub. No.: US 2025/0195654 A1
(43) Pub. Date: Jun. 19, 2025

(54) TUMOR MICROENVIRONMENT-REGULATED CAR-MONOCYTE/MACROPHAGE, AND PREPARATION METHOD THEREFOR AND USE THEREOF

(71) Applicant: Xidian University, Xi'an (CN)

(72) Inventor: Pengbo NING, Xi'an (CN)

(73) Assignee: Xidian University, Xi'an (CN)

(21) Appl. No.: 18/845,856

(22) PCT Filed: Jan. 30, 2023
(86) PCT No.: PCT/CN2023/073798

§ 371 Date: Sep. 10, 2024

(30) Foreign Application Priority Data
Mar. 11, 2022 (CN) 202210237581.5

(51) Int. Cl. A61K 40/17; A61K 40/31; A61K 40/35; A61K 40/42; A61P 35/00; C07K 14/535; C07K 16/32; C12N 5/0786

(52) U.S. Cl. CPC A61K 40/17; A61K 40/31; A61K 40/35; A61K 40/4205; A61P 35/00; C07K 14/535; C07K 16/32; C12N 5/0645; A61K 2239/49; C07K 2317/622; C07K 2319/03; C12N 2510/00

(57) ABSTRACT

The present invention belongs to the technical fields of immunology and oncologic therapies. Provided are a tumor microenvironment-regulated CAR-monocyte/macrophage, and a preparation method therefor and the use thereof. When the CAR-monocyte/macrophage forms a chimeric antigen receptor composite structure, GM-CSF can be expressed intracellularly and autocrine to extracellular to promote the differentiation of the CAR-monocyte/macrophage to form an M1 type macrophage, so that the tumor microenvironment can be further regulated while the property of resisting M2 type macrophage reversal in the tumor microenvironment is maintained, thereby sensitizing the anti-tumor effect of the CAR-monocyte/macrophage. On the basis of GM-CSF, a tumor microenvironment-regulated CAR macrophage technology platform is constructed, which can maintain the M1-type characteristics of CAR-M and can also produce a TME reversal effect, thereby achieving the efficient anti-tumor effect of a CAR-monocyte/macrophage.

### CROSS-REFERENCE TO RELATED APPLICATIONS

The application claims priority to Chinese patent application No. 2022102375815, filed on Mar. 11, 2022, the entire contents of which are incorporated herein by reference.

### SEQUENCE LISTING

The sequence listing xml file submitted herewith, named “PCT2301003.xml”, created on Aug. 30, 2024, and having a file size of 14,672 bytes, is incorporated by reference herein.

### TECHNICAL FIELD

The present invention relates to the technical field of immunology and tumor therapeutics, and particularly, to a tumor microenvironment-regulatory CAR-monocyte/macrophage, a method for preparing same, and use thereof.

### BACKGROUND

In recent years, anti-tumor cell immunotherapy has been widely applied to studies and clinical practice for various malignant tumors, and especially, CAR-T cell immunotherapy exhibits good efficacy in treating hematological tumors. In solid tumors, however, the suppressive tumor microenvironment (TME) typically forms an immune barrier to adoptive cell immunotherapy by a variety of factors. The tight cellular and stromal barrier forms a physical barrier to the engineered lymphocytes infiltrating into solid tumors. Even when infiltrating into solid tumors, the immunosuppressive barrier formed by TME, for example, hypoxic and acidic environments, the expression of immune checkpoint ligands, and particularly, the massive immunosuppressive cells such as tumor-associated macrophages (TAMs), myeloid-derived suppressor cells and regulatory T cells, are one of the major challenges in current solid tumor immunotherapy.

The immunosuppressive mechanism of TME is complicated, and one of the important factors is the immunosuppressive mechanism of tumor-associated macrophages (TAMs). Tumors recruit circulating monocytes and tissue resident macrophages to TME through various soluble and mechanical factors and polarize them into the M2 phenotype, thereby producing TAMs. TAMs, as compared with macrophages in physiological conditions, remarkably vary in immune recognition and phagocytotic capacity for tumor cells, and lose their basic immune functionality for recognizing abnormal cells and eliminating immunogens. Meanwhile, TAMs play a key role in inhibiting endogenous anti-tumor immunity by up-regulating immunosuppressive surface proteins, secreting active oxygen, producing cytokines for inhibiting T cell functionality, and secreting chemokines for recruiting Treg cells. In addition, TAMs promote tumor angiogenesis and metastasis by secreting factors such as VEGF and matrix metalloproteinases, which can rebuild the TME, increase angiogenesis, and promote tumor cell migration. Since macrophage polarization is a progressive process, as the oncosuppressive signals increase in the tumor microenvironment, TME may promote the conversion of more recruited macrophages in solid tumors from the M1 phenotype to the M2 TAM phenotype. As tumors become more malignant, TAMs may reach a proportion of 50% or higher within the solid tumors.

For the immunosuppressive properties of TAMs, a part of current clinical immunotherapies adopt different strategies such as inhibiting the recruitment of macrophages into solid tumors, killing macrophages, inducing the repolarization of M2 macrophages to the M1 phenotype, or promoting the reprogramming of TAMs to the M1 phenotype. Some studies attempted to block the CSF1 (also known as MCSF)/CSFIR axis, an important ligand-receptor pair for macrophage differentiation and survival, so as to block monocyte differentiation, thus reducing the number of TAMs and decreasing the viability of existing TAMs. Additionally, in some studies, small molecule inhibitors of CSFIR were used to induce the repolarization of TAM from the M2 to the M1 phenotype, thereby restoring the anti-tumor functionality of TAMs. Such strategies still focus on the amount of TAMs to improve the efficacy.

For the challenges in immunotherapies against solid tumors, the chimeric antigen receptor-macrophage (CAR-M) immunotherapy has become a novel adoptive cell immunotherapy technology developed in recent years. Constructing CARs using a monocyte/macrophage provides a possible solution for overwhelming solid tumors. CAR-M cells can specifically gather at solid tumors and penetrate the dense interstitial tissue surrounding the tumor. Moreover, the CAR-M combines a direct tumoricidal approach with the ability to enhance endogenous immunity through antigen presentation, making it a unique path for the development of anti-tumor cell therapies. It was found that CAR-M can drive the phagocytosis and killing of target tumor cells without the addition of soluble conditioning factors. Morrissey, et al., found that the construction of mouse macrophage lines and mouse bone marrow-derived macrophages using anti-CD19 and anti-CD22 CARs mediated the phagocytosis of antigenic beads and tumor cells, confirming the ability of CARs to induce the phagocytic pathway. Zhang, et al., induced the expression of CARs in induced pluripotent stem cells (iPSCs) and induced the differentiation of CAR-iPSC into CAR-iMac. In the absence of antigen stimulation, the CAR-iMac is closer to the M2 phenotype. Upon antigenic stimulation, the CAR-mediated signals promote the phagocytosis of CAR-iMac.

Although the novel CAR-M technology is clearly advantageous in addressing solid tumors, the therapeutic effect of CAR-M, as an adoptive immunotherapy using the macrophage system, is often hindered by the suppressive TME inside solid tumors through a variety of factors, and the CAR-M also poses the risks of polarization into M2 macrophages after infiltrating into the solid tumors. How to avoid the development of TAM-related carcinogenic characteristics in the reintroduced macrophage is an important problem to be solved in the development of CAR-M technology. As such, an ideal design should be able to both maintain the M1 characteristics of the CAR-M and generate a TME-reversing effect. The design of specific elements for the CAR-M signaling functionality offers the possibility to improve the anti-tumor potential of the CAR-M.

### SUMMARY

The present invention is intended to provide a tumor microenvironment-regulatory CAR-monocyte/macrophage, a method for preparing same, and use thereof to break through the bottleneck in the development of CAR-M in the prior art. By designing and constructing the tumor microenvironment-regulatory TMER CAR (monocyte) macrophage on the basis of the GM-CSF motif, the CAR-M scFv, after targeting and killing tumor cells, can retain its M1 phenotype in the tumor microenvironment, achieve re-education of TAMs in the TME, and reverse the tumor microenvironment, thus achieving a better anti-tumor effect.

For the above purpose, the present invention provides the following embodiments:

The present invention provides a tumor microenvironment-regulatory CAR-monocyte/macrophage, wherein the CAR-monocyte/macrophage is a complex structure comprising a chimeric antigen receptor capable of producing GM-CSF expressed intracellularly and secreted extracellularly to promote the differentiation of the CAR-monocyte/macrophage to an M1 macrophage and further regulate the tumor microenvironment; the GM-CSF comprises the nucleotide sequence set forth in SEQ ID NO: 1 or SEQ ID NO: 9.

The present invention further provides a chimeric antigen receptor, comprising GM-CSF, a HER2 scFv, a transmembrane region of α1β1 integrin, an intracellular region of α1β1 integrin, and an intracellular region of FcγRI transmembrane signaling domain.

Preferably, the nucleotide sequences of the GM-CSF, the HER2 scFv, the transmembrane region of α1ß1 integrin, the intracellular region of α1ß1 integrin, and the intracellular region of FcγRI transmembrane signaling domain are set forth in SEQ ID NO: 1/SEQ ID NO: 9, SEQ ID NO: 2, SEQ ID NO: 3/SEQ ID NO: 10, SEQ ID NO: 4/SEQ ID NO: 11, and SEQ ID NO: 5/SEQ ID NO: 12, respectively.

The present invention further provides a recombinant vector, comprising the chimeric antigen receptor.

The present invention further provides a method for preparing the tumor microenvironment-regulatory CAR-monocyte/macrophage, comprising: constructing the recombinant vector, and transfecting the CAR-monocyte/macrophage with the recombinant vector.

Preferably, constructing the recombinant vector comprises:

(1) constructing the chimeric antigen receptor: connecting the HER2 scFv, the transmembrane region of α1β1 integrin, the intracellular region of α1β1 integrin, and the intracellular region of FcγRI transmembrane signaling domain via a linker, and connecting the GM-CSF via a P2A to synthesize the chimeric antigen receptor; and

(2) recombining the chimeric antigen receptor with a lentiviral vector to construct the recombinant vector.

The present invention further provides a pharmaceutical composition, comprising: the CAR-monocyte/macrophage, the chimeric antigen receptor or the recombinant vector, and a pharmaceutically acceptable excipient or carrier.

The present invention further provides use of the CAR-monocyte/macrophage in preparing a medicament for promoting the differentiation to an M1 macrophage.

The present invention further provides use of the CAR-monocyte/macrophage in preparing a medicament for treating a solid tumor, wherein the solid tumor may be selected from, but is not limited to, a solid tumor with high HER2 expression.

The present invention discloses the following technical effects:

The present invention designs and constructs a tumor microenvironment-regulatory TMER CAR (monocyte) macrophage (TMER CAR-M) on the basis of the GM-CSF motif, and based on the technical platform, the GM-CSF motif and the chimeric antigen receptor form a complex structure. The constructed CAR-M can secrete GM-CSF extracellularly, leading to a higher level of inflammatory anti-tumor cytokines. As such, CAR-M scFV can retain the M1 macrophage phenotype and avoid being converted by the tumor microenvironment while targeting and killing tumor cells. Also, GM-CSF can infiltrate into the TME to regulate the tumor microenvironment and enhance the anti-tumor effect.

The present invention designs a polarization element suitable for macrophage functions on the basis of GM-CSF motif, and thereby designs and implements a tumor microenvironment-regulatory TMER CAR-M (TMER CAR-M) cell technology platform. The platform remarkably improves the anti-tumor characteristics of the CAR-M and plays a role in maintaining the M1 phenotype and regulating and controlling the tumor microenvironment, and is thus suitable for the application and extension design of various types of CAR-M in treating solid tumors in the future.

### BRIEF DESCRIPTION OF DRAWINGS

In order to more clearly illustrate the technical solutions in the embodiments of the present invention or the prior art, drawings for illustrating the embodiments are briefly described below. Apparently, the drawings in the following description only illustrate some embodiments of the present invention. For those of ordinary skill in the art, other drawings can be obtained according to such drawings without creative efforts.

FIG. 1 illustrates the construction and characterization of macrophage-specific activating TMER CAR-M according to the present invention, where A: transmembrane expression of TMER CAR by confocal microscopy; B: expression of TMER CAR by qPCR; C: HER2 binding capacity of TMER CAR-M by confocal microscopy, in a scale size of 30 μm;

FIG. 2 illustrates an activity assay of the TMER CAR-M according to the present invention, where A: the proliferation of TMER CAR-M by CCK-8; B: the expression of p53 by qPCR; C: the expression of CCND-1 by qPCR; D: the expression of Ki67 by qPCR; * denotes significant differences among different groups, *** denotes P<0.001, ** denotes P<0.01, and * denotes P<0.05;

FIG. 3 illustrates an M1 phenotypic polarization assay of the TMER CAR-M according to the present invention in vitro, where A: the expression of CD80 by qPCR; B: the expression of IFN-γ by qPCR; C: the expression of iNOS by qPCR; D: the expression of CD206 by qPCR; E: the expression of Arg-1 by qPCR; F: the expression of IL-10 by qPCR; * denotes significant differences among different groups, *** denotes P<0.001, ** denotes P<0.01, and * denotes P<0.05;

FIG. 4 illustrates an in vitro M2 polarization resistance assay of the TMER CAR-M according to the present invention, where A: the expression of CD80 by qPCR; B: the expression of IFN-γ by qPCR; C: the expression of iNOS by qPCR; D: the expression of CD206 by qPCR; E: the expression of Arg-1 by qPCR; F: the expression of IL-10 by qPCR; * denotes significant differences among different groups, *** denotes P<0.001, ** denotes P<0.01, and * denotes P<0.05;

FIG. 5 illustrates an analysis of TMER CAR-M regulating the M2 macrophage reversion in vitro according to the present invention, where A: the expression of CD206 by qPCR; B: the expression of Arg-1 by qPCR; C: the expression of IL-10 by qPCR; D: the expression of CD80 by qPCR; E: the expression of IFN-γ by qPCR; F: the expression of iNOS by qPCR; * denotes significant differences among different groups, *** denotes P<0.001, ** denotes P<0.01, and * denotes P<0.05;

FIG. 6 illustrates the anti-tumor results of TMER CAR-M verified in a mouse HER2+-4T1 tumor model according to the present invention, where A: the TMER CAR-M treatment in the HER2+-4T1 tumor-bearing mice; B. tumor growth curves in mice receiving the TMER CAR-M treatment; C. survival curves of mice receiving the TMER CAR-M treatment; D. body weight of mice receiving the TMER CAR-M treatment; * denotes significant differences among different groups, *** denotes P<0.001, ** denotes P<0.01, and * denotes P<0.05;

FIG. 7 illustrates the results of monitoring the anti-tumor effects of TMER CAR-M in nude mouse SKOV3 (human ovarian cancer cell) peritoneal tumor model by an imaging system IVIS Lumina II.

### DETAILED DESCRIPTION OF THE EMBODIMENTS

Various exemplary embodiments of the present invention are described in detail below, which should not be construed as limitations to the present invention but as a more detailed description of certain aspects, features, and embodiments of the present invention.

It will be appreciated that the terminology used herein is for the purpose of illustrating particular embodiments only, rather than limiting the present invention. In addition, for numerical ranges in the present invention, it will be appreciated that each intervening value, to the upper and lower limits of the range, is also specifically disclosed. Every smaller range between any stated value or intervening value in a stated range and any other stated or intervening value in that stated range is encompassed within the present invention. The upper and lower limits of such smaller ranges may independently be included or excluded in the range.

Unless defined otherwise, all technical and scientific terms used herein have the same meaning as commonly understood by those of ordinary skill in the art. Although only preferred methods and materials are described herein, any methods and materials similar or equivalent to those described herein can be used in the practice or testing of the present invention. All documents described herein are incorporated by reference to disclose and describe the methods and/or materials associated with the documents. In case of conflict with any incorporated document, the specification shall prevail.

It will be apparent to those skilled in the art that various modifications and variations can be made to the specific embodiments of the present invention without departing from the scope or spirit of the present invention. Other embodiments will be apparent to those skilled in the art from consideration of the specification. The specification and examples are exemplary only.

As used herein, the terms “comprise”, “include”, “have”, “contain”, and the like are open-ended terms that mean including but not limited to.

### Example 1 Preparation and Functional Verification of Tumor Microenvironment-Regulatory CAR (Monocyte) Macrophages

### 1. Construction and Characterization of Tumor Microenvironment-Regulatory TMER CAR-M

According to the extracellular binding principle of CAR-T and the budding mechanism of enveloped viruses, a lentivirus vector was adopted to construct the macrophage-specific CAR and the tumor microenvironment-regulatory TMER CAR-M, and a tumor microenvironment-regulatory TMER CAR-M was established with HER2 as the model molecule. A transmembrane HER2 scFv extracellular segment was expressed on the surface of cell membranes, and stable TMER CAR-M cell lines with high HER2-binding affinity and activity were obtained through screening.

### 1.1 Construction and Packaging of Lentiviruses

Based on the lentiviral vectors, a HER2 scFv, the transmembrane region of α1β1 integrin, the intracellular region of α1β1 integrin, the intracellular region of FcγRI transmembrane signaling domain, and GFP were connected via a linker, and the GM-CSF motif was connected via P2A, so as to synthesize the chimeric antigen receptor HER2 scFv-GM-CSF (sequence synthesized by Sangon Biotech (Shanghai) Co., Ltd.). Macrophages were transfected with the sequence to construct a macrophage-specific CAR by co-transfection of the synthesized HER2 scFv-GM-CSF-CAR lentivirus plasmid with three helper plasmids Rev, Gag and VSV, and packaging into a complete HER2 scFv-GM-CSF-CAR lentivirus.

The specific procedures are as follows:
(1) 4×10⁵ HEK293T cells in the logarithmic phase were uniformly seeded on a 6-well plate and cultured in an incubator at 37° C. and 5% CO₂ until the cell confluence reached 60%-70%. Plasmids pCDH-HER2 scFv-GM-CSF, Rev, Gag, and VSV were added into a centrifuge tube containing Opti-MEM in a ratio of 3:1:1:1. The mixture was uniformly mixed and incubated at room temperature before a TurboFect Transfection Reagent was added. The mixture was then uniformly mixed by pipetting, and incubated at room temperature;
(2) The incubated mixture was slowly and dropwise added into the 6-well plate containing the 293T cells with shaking. The plate was incubated in an incubator at 37° C. and 5% CO2 for 16-24 h. The transfection mixture in each well of the 6-well plate was discarded before 2-3 mL of Advanced DMEM complete culture medium containing 10% fetal bovine serum (FBS) was added. The cell culture plate was cultured in an incubator at 37° C. and 5% CO2 for 48 h for the synthesis of HER2 scFv-GM-CSF-CAR lentivirus;
(3) Cell debris was removed, and the lentivirus-containing medium was centrifuged at 4000 g for 10 min at 4° C. The supernatant was filtered through a 0.22-μm filter, and the filtrate was collected and centrifuged at 15000 g for 2 h at 4° C. The supernatant was discarded. The virus particles were resuspended in a virus preservation solution and centrifuged at 10000 g for 5 min. The supernatant was preserved at −80° C. for later use;
(4) The titer of the packaged lentiviruses was then determined: 90 μL of DMEM complete culture medium was added to 9 sterile 1.5-mL centrifuge tubes, before 10 μL of the obtained recombinant lentivirus particles HER2 scFv-GM-CSF-CAR was added into the first centrifuge tube. The mixture was uniformly mixed by pipetting, and 10 μL of the mixture was transferred into the second centrifuge tube. The procedures were repeated until the last tube, and the groups were in triplicate. 5×104 HEK293T cells were uniformly seeded on a 96-well plate. The lentivirus dilutions were added when the cell confluency reached 60%-70%. 293T cells without virus dilutions were used as the negative control. After 24 h of incubation, the medium was replaced with a fresh DMEM complete medium, and after 48 h, the cells were observed under an inverted fluorescence microscope. The lentivirus titer was calculated according to the formula: viral titer (TU/mL)=mean count of green fluorescent cells× dilution factor/volume of virus dilution (mL), in TU/mL.

The sequences of the elements for constructing the chimeric antigen receptor are as follows:

Sequence of murine GM-CSF (SEQ ID NO: 1):
atgtggctgcagaacctgctgttcctgggcatcgtggtgtacagc
ctgagcgcccccacaaggtctccaatcacagtgaccagaccctg
gaagcacgtggaggccatcaaggaggccctgaatctgctggacga
catgcccgtgaccctgaacgaagaggtggaggtggtgtccaacga
gttcagcttcaagaagctgacctgtgtgcagaccaggctgaagat
cttcgagcagggcctgaggggcaacttcaccaagctgaagggagc
cctgaatatgaccgccagctactaccagacctactgcccccccac
ccctgagacagattgcgagacccaggtgaccacctacgccgactt
catcgatagcctcaagacctttctgacagacatccccttcgagtg
taagaagccaggccagaag.
Sequence of HER2 scFv (SEQ ID NO: 2):
atgaatttacaaccaattttctggattggactgatcagttcagtt
tgctgtgtgtttgctctagcttctcaggtacaactgcagcagtc
tggacctgaactgaagaagcctggagagacagtcaagatctcctg
caaggcctctgggtatcctttcacaaactatggaatgaactgggt
gaagcaggctccaggacagggtttaaagtggatgggctggattaa
cacctccactggagagtcaacatttgctgatgacttcaagggacg
gtttgacttctctttggaaacctctgccaacactgcctatttgca
gatcaacaacctcaaaagtgaagacatggctacatatttctgtgc
aagatgggaggtttaccacggctacgttccttactggggccaagg
gaccacggtcaccgtttcctctggcggtggcggttctggtggcgg
tggctccggcggtggcggttctgacatccagctgacccagtctca
caaattcctgtccacttcagtaggagacagggtcagcatcacctg
caaggccagtcaggatgtgtataatgctgttgcctggtatcaaca
gaaaccaggacaatctcctaaacttctgatttactcggcatcctc
ccggtacactggagtcccttctcgcttcactggcagtggctctgg
gccggatttcactttcaccatcagcagtgtgcaggctgaagacct
ggcagtttatttctgtcagcaacattttcgtactccattcacg.
Sequence of transmembrane region of murine
α1β1 integrin (SEQ ID NO: 3):
ttatgggtcatcctgctgagtgcttttgccggattgttgctgtta
atgctgctcattttagcactgtgg.
Sequence of intracellular region of murine
α1β1 integrin (SEQ ID NO: 4):
aagatcggattcttcaagagacctctgaagaagaagatggagaag.
Sequence of intracellular region of murine
FcγRI transmembrane signaling domain
(SEQ ID NO: 5):
aagatccatagactgcagagagagaagaagtacaacctggaggtg
cctctggtgagcgagcagggcaagaaggccaacagcttccagca
ggtgaggagcgacggcgtgtacgaggaggtgaccgccaccgccag
ccagaccacccctaaggaggcccctgacggccctaggagcagcgt
gggcgactgtggccctgagcagcctgagcctctgcctcctagcga
cagcaccggcgcccagaccagccagagc.
Sequence of GFP (SEQ ID NO: 6):
gtgagtaagggcgaggagctgtttaccggcgtggtgcccatcctg
gtggagctggacggcgacgtgaacggccacaagttcagcgtgag
cggcgagggggagggcgacgccacctacggcaagctgaccctgaa
gttcatttgcacaaccggcaagctgcccgtgccatggcctaccct
ggtgaccaccctgacatacggcgtccagtgttttagcaggtatcc
cgaccacatgaagcagcacgacttctttaagagcgccatgcccga
gggctacgtgcaggagaggaccatcttcttcaaggacgacggtaa
ctacaagaccagagccgaggtgaagttcgagggcgacaccctggt
gaaccggatcgagctgaagggcatcgacttcaaggaggacggtaa
catcctgggccacaagctggagtataactataatagccacaacgt
gtacatcatggccgacaagcagaagaacggcattaaggtgaactt
caagatcagacataacatcgaggacggatctgtgcagctggccga
ccactaccagcagaacacccctatcggagatggaccagtgctgct
gcctgataatcactatctgagcacacagtctgccctgagcaaaga
tcctaatgaaaagagagatcacatggtgctgctggagtttgtgac
agctgctggaataacactgggcatggatgagctgtataagtaa.
Sequence of linker (SEQ ID NO: 7):
ggcggtggttccggcggtggatctggtggaggaactggaggaggt
tcaggaggtggt.
Sequence of P2A (SEQ ID NO: 8):
ggaagcggagctactaacttcagcctgctgaagcaggctggagac
gtggaggagaaccctggacct.
Sequence of human GM-CSF (SEQ ID NO: 9):
atgtggctgcagtccctgctgctgctggggacagtggcctgcagc
atcagcgcccccgccaggagccccagccccagcacacagccctg
ggagcacgtgaacgccatccaggaggcccggcggctgctgaacct
gagcagggataccgccgccgagatgaacgagaccgtggaggtcat
cagcgagatgtttgacctgcaggagcccacatgcctgcagacccg
gctggagctgtacaagcagggcctgagggggtccttgacaaagct
gaagggccccctgacaatgatggccagccactacaagcagcactg
cccccccacccccgagacctcctgcgccacccagatcatcacctt
cgagagcttcaaggagaacctgaaggatttcctgctcgtgatccc
cttcgactgctgggagcccgtgcaggag
Sequence of transmembrane region of human
α1β1 integrin (SEQ ID NO: 10):
ctgtgggtcatcctgctgagcgccttcgccgggctgctgctgctg
atgctgctgatcctggccctgtgg
Sequence of intracellular region of human
α1β1 integrin (SEQ ID NO: 11):
aagatcggcttctttaagcggcccctgaagaagaagatggagaag
Sequence of intracellular region of human
FcγRI transmembrane signaling domain
(SEQ ID NO: 12):
cggaaggagctgaagaggaagaagaagtgggacctggagatcagc
ctggatagcggccacgagaagaaggtcatctccagcctgcagga
ggataggcacctggaggaggagctgaagtgccaggagcagaagga
ggagcagctgcaggagggggtgcaccggaaggagccccagggcgc
caca

### 1.2 Preparation of TMER CAR-M Cells

After packaging into an intact lentivirus and the titer measurement, the macrophages were infected with the HER2 scFv-GM-CSF-CAR lentivirus to give a stably transfected TMER CAR-M cell line. The macrophages were seeded on a 6-well plate and cultured until a confluency of 60-70%. The HER2 scFv-GM-CSF-CAR lentiviral particles were added to the macrophage culture in a ratio of 9:1. The medium was replaced with a fresh complete DMEM culture medium at about 24 h, and at about 48 h, the lentivirus infection of the macrophages was completed. The growth condition and morphological changes of the cells were observed, and the cells were transferred to a new culture flask or a new culture dish for expansion or cryopreservation.

1.3 qPCR and Confocal Microscopic Examination of Constructed TMER CAR-M

The total RNA of the stably transfected cell line was extracted. The expression of the fusion protein in the stably transfected cells was determined using a SuperReal PreMix Plus (SYBR Green) kit according to the kit instructions. The qPCR procedures are as follows:
(1) 2× SuperReal Premix Plus, 50×ROX Reference Dye, templates, and primers were dissolved in RNase-free ddH2O. The reagents were equilibrated at room temperature and mixed well;
(2) The reversely transcripted cDNA template in a certain system was mixed with the sense primer, antisense primer, Mix, ROX, and ddH2O on ice. The mixture was added to eight tubes using a pipette. The 20-μL system contained: 10 μL of 2× SuperReal Premix Plus, 0.6 μL of sense primer (10 μM), 0.6 μL of antisense primer (10 μM), 0.1-2 μL of cDNA template, 0.4 μL of 50×ROX Reference Dye, and the remaining of RNase-free ddH2O;
(3) The tubes were capped, uniformly mixed, and centrifuged for 5-10 s on a microcentrifuge to precipitate all components at the bottom of the tube;
(4) The reaction system was loaded on an RT-qPCR system. The parameters were set to: pre-denaturation at 95° C. for 15 min, denaturation at 95° C. for 10 s, annealing at 60±1° C. for 20 s, extension at 72° C. for 31 s, and 40 cycles. The procedures were conducted, a Ct value was determined by derivation after the program was finished, and the final result was calculated by the 2-ΔΔCt (Livak) method.

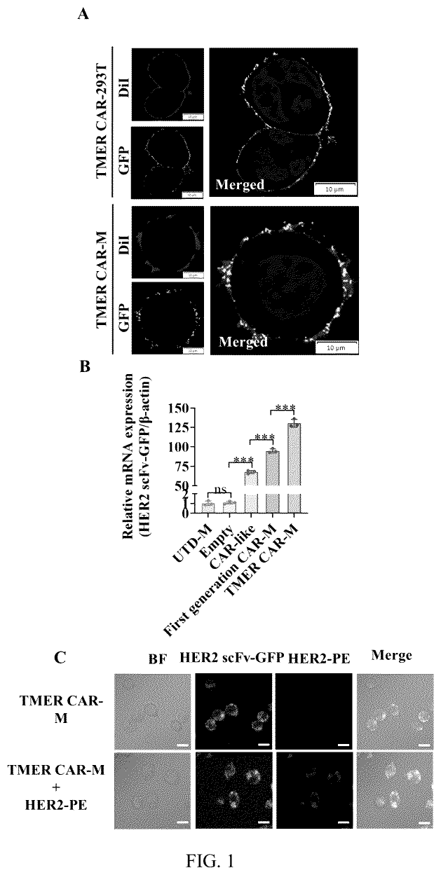

As shown in FIG. 1A, DiI in TMER CAR-293T and TMER CAR-M denotes the positions on cell membranes, GFP denotes the expressed transmembrane CAR structure, and Merged denotes the overlay of the two. The confocal microscopic results show that the CAR structure according to the present invention was successfully expressed on the cell membrane; as shown in FIG. 1B, further qPCR quantitative analysis verified the significant high expression of the CAR sequence in the corresponding group, suggesting the successful construction of the TMER CAR-M. Among the confocal microscopic results shown in FIG. 1C, BF in TMER CAR-M denotes the bright field photographing results, HER2 scFv-GFP denotes the TMER CAR-M, HER2-PE denotes the PE-labeled HER2 tumor antigen protein, and Merge represents the overlay of the three, showing that TMER CAR-M achieves efficient binding to HER2 through the expression of transmembrane HER2 scFv. The results shown in FIG. 1 demonstrate the successful expression of the TMER CAR-M fusion protein, which indicates the successful construction of the TMER CAR-M having significant HER2 tumor antigen binding activity, providing a cell therapy model for future studies.

### 2. Activity Assay of TMER CAR-M

### (1) CCK Cell Proliferation Assay

A. Macrophage suspensions at a density of 1× 104 cells/mL of different groups, J774A.1 (UTD-M), Empty-pCDH J774A. 1 (Empty), HER2 scFv J774A.1 (CAR-like), HER2 scFv CAR J774A.1 (First generation CAR-M), and HER2 scFv-GM-CSF CAR J774A.1 (TMER CAR-M), were added to a 96-well plate at 100 μL/well. The cells were cultured in an incubator at 37° C. and 5% CO2;
B. After the cells were cultured for 24-48 h, the culture medium in a 96-well plate was discarded. The cells were washed twice or thrice with PBS before the CCK8 detection solution was added to the 96-well plate at 10 μL/well. The cells were cultured for 3-4 h in an incubator with 37° C. and 5% CO2;
C The absorbance of the wells at 450 nm was determined on a microplate reader, and the cell activity of the target cells=(absorbance of effector/target action group-absorbance of effector cell control group)/(absorbance of target cell positive control group-absorbance of blank well).

(2) The total RNA of each group was extracted, and the activity of the TMER CAR-M was determined by qPCR according to the same qPCR method as described above.

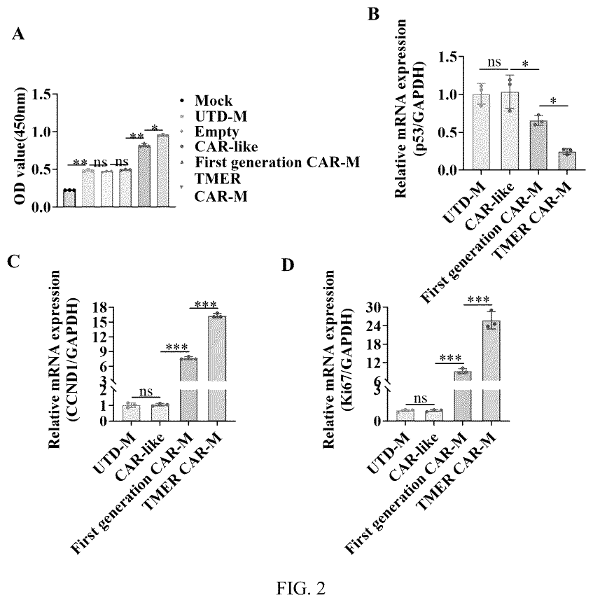

As shown in FIG. 2, the cell proliferation in the TMER CAR-M group was significantly enhanced over the CAR-Like group and First generation CAR-M group (FIG. 2A). The qPCR results show that the mRNA expression levels of proliferation factors CCND1 and Ki67 in the TMER CAR-M group were significantly higher than those of the CAR-Like group and First generation CAR-M group, while the expression level of p53 was lower than those of the CAR-Like group and the First generation CAR-M group, as shown in FIGS. 2B-2E. The results of the study indicate that the basal viability of the TMER CAR-M is significantly enhanced, suggesting that the expression of GM-CSF enhances the viability of TMER CAR-M cells.

### 3. In Vitro M1 Phenotype Polarization Assay of TMER CAR-M

The TMER CAR-M cells constructed as described above were separately cultured and tested in 6-well plates based on a density of 2×105 cells/mL. The specific procedures are as follows:

UTD-M, Empty, CAR-like, First generation CAR-M, and TMER CAR-M were separately seeded on a 6-well plate at 4×10⁵ cells/well and cultured in a DMEM high-sugar medium containing 10% FBS in an incubator at 37° C. and 5% CO2 for 48 h. The total RNA of each group was separately extracted, and the TMER CAR-M polarization was determined by qPCR according to the same qPCR method as described above.

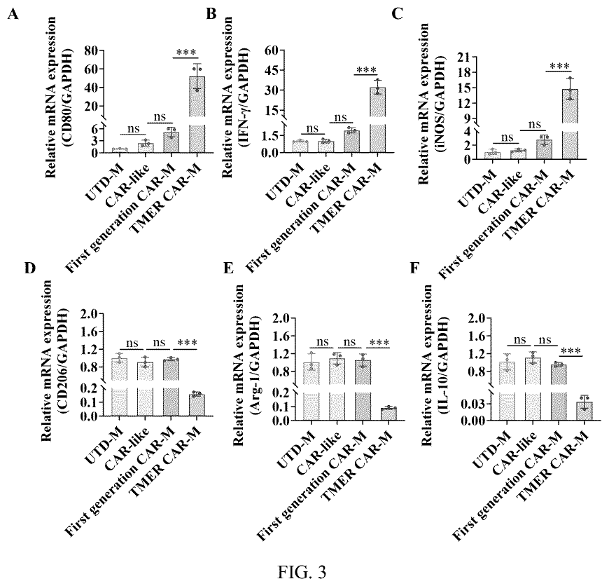

The results, as shown in FIG. 3, show that the TMER CAR-M group showed significantly elevated expression levels of M1 factors CD80, IFN-γ, and iNOS, but significantly reduced expression levels of M2 factors CD206, Arg-1, and IL-10, compared with the CAR-like group and First generation CAR-M group, indicating that the TMER CAR-M group exhibited significant anti-tumor characteristics of M1 macrophages relative to both the CAR-like group and the First generation CAR-M group. This suggests that GM-CSF plays a role in the polarization of TMER CAR-M and that the TMER CAR-M may be resistant to M2 polarization.

### 4. In Vitro M2 Polarization Resistance Validation of TMER CAR-M

A tumor cell culture supernatant was adopted to simulate the suppressive tumor microenvironment system in vitro. The TMER CAR-M, CAR-Like, and a normal macrophage control group were examined. The successfully constructed TMER CAR-M cells were compared with the CAR-Like and First generation CAR-M groups. The TMER CAR-M cells were induced in the tumor culture supernatant for 5 days, and the total RNA was then extracted and analyzed by qPCR for the anti-polarization capability of the TMER CAR-M cells. The specific procedures are as follows:
(1) UTD-M, CAR-like, First generation CAR-M, and TMER CAR-M cells were added into 6-well plates at 2×10⁵ cells/well, and cultured in an incubator at 37° C. and 5% CO₂;
(2) When the UTD-M, CAR-like, First generation CAR-M, and TMER CAR-M cells adhered to the flask wall, the culture medium in the plates was discarded. The cells were washed twice or thrice with PBS, and the tumor cell culture supernatant was added to the 6-well plates. The groups were in triplicate.
(3) After 5 days of cell induction, the culture medium in the 6-well plates was discarded. The cells were washed twice or thrice with PBS, before Trizol was added to the 6-well plates at 1 mL/well. The RNA was extracted using a SuperReal PreMix Plus (SYBR Green) kit according to the kit instructions.
(4) 2× SuperReal Premix Plus, 50×ROX Reference Dye, templates, and primers were dissolved in RNase-free ddH₂O. The reagents were equilibrated at room temperature and mixed well;
(5) The reversely transcripted cDNA template in a certain system was mixed with the sense primer, antisense primer, Mix, ROX, and ddH₂O on ice. The mixture was added to eight tubes using a pipette. The 20-μL system contained: 10 μL of 2× SuperReal Premix Plus, 0.6 μL of sense primer (10 μM), 0.6 μL of antisense primer (10 μM), 0.1-2 μL of cDNA template, 0.4 μL of 50×ROX Reference Dye, and the remaining of RNase-free ddH₂O;
(6) The tubes were capped, uniformly mixed, and centrifuged for 5-10 s on a microcentrifuge to precipitate all components at the bottom of the tube;
(7) The reaction system was loaded on an ABI 7300 RT-qPCR system. The parameters were set to: pre-denaturation at 95° C. for 15 min, denaturation at 95° C. for 10 s, annealing at 60±1° C. for 20 s, extension at 72° C. for 31 s, and 40 cycles. The procedures were conducted, a Ct value was determined by derivation after the program was finished, and the final result was calculated by the 2^{-ΔΔCt} (Livak) method.

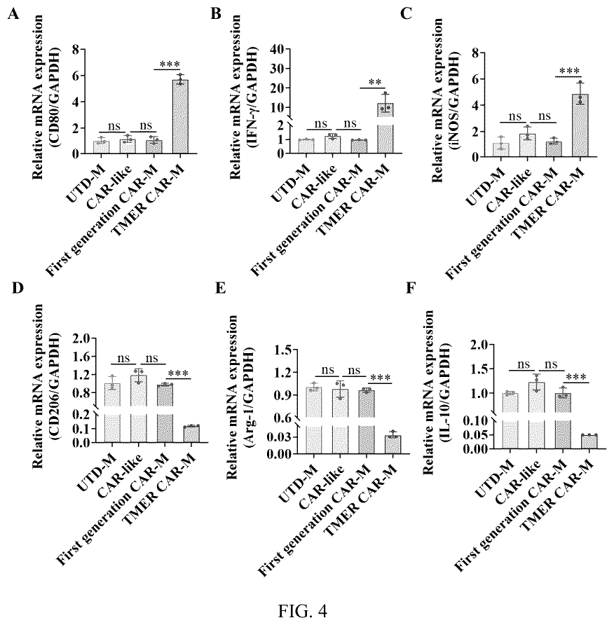

The results, as shown in FIG. 4, show that under the effect of the tumor supernatant, the expression levels of M2 factors CD206, Arg-1, and IL-10 in the CAR-like group, First generation CAR-M group, and normal macrophage group were significantly increased compared with the TMER CAR-M group (as shown in FIGS. 4E and 4F), indicating that in the CAR-like group, First generation CAR-M group, and normal macrophage group, the tumor microenvironment induces the M2 polarization, while the TMER CAR-M group still retains significant M1 macrophage characteristics.

### 5. TMER CAR-M Regulating M2 Macrophage Reversion to M1 Macrophage In Vitro

A tumor cell culture supernatant was adopted to simulate the suppressive tumor microenvironment system in vitro. The TMER CAR-M, CAR-Like, and a normal macrophage control group were examined. The successfully constructed TMER CAR-M cells were compared with the CAR-Like and First generation CAR-M groups. Normal macrophages were induced in the tumor culture supernatant and then induced in a TMER CAR-M cell culture supernatant, and the total RNA was then extracted and analyzed by qPCR for the anti-polarization capability of the TMER CAR-M cells. The specific procedures are as follows:
(1) Normal macrophages were added into a 6-well plate at 2×10⁵ cells/well and induced for 5 days in the tumor cell culture supernatant by incubation in an incubator at 37° C. and 5% CO₂. The groups were in triplicate;
(2) After 5 days of cell induction, the culture medium in the 6-well plates was discarded. The cells were washed twice or thrice with PBS and induced in UTD-M, CAR-like, First generation CAR-M, or TMER CAR-M cell culture supernatant;
(3) After 5 days of cell induction, the culture medium in the 6-well plates was discarded. The cells were washed twice or thrice with PBS, before Trizol was added to the 6-well plates at 1 mL/well. The RNA was extracted and detected by qPCR according to the same qPCR method as described above.

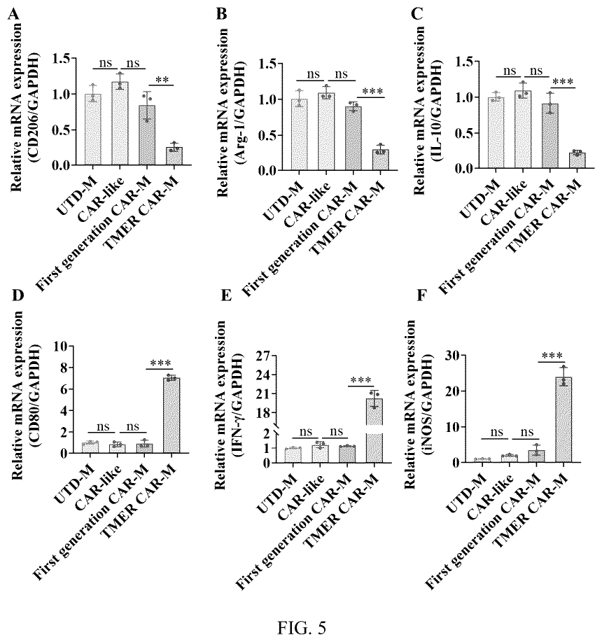

The results, as shown in FIGS. 5A-5C, show that, compared with the CAR-like group, First generation CAR-M group, and normal macrophage group, under the effect of the TMER CAR-M supernatant, the expression levels of M2 factors CD206, Arg-1 and IL-10 in the CAR-like group, First generation CAR-M group, and normal macrophage group were significantly increased, suggesting that the CAR-like group, the First generation CAR-M group, and the general macrophage group cannot induce the reprogramming of M2 macrophage to the M1 phenotype in the tumor microenvironment, while the macrophages induced by the TMER CAR-M supernatant exhibited significant M1 macrophage characteristics. This indicates that the TMER CAR-M supernatant can regulate the reversion of M2 macrophages to the M1 phenotype, preliminarily confirming the in vitro tumor microenvironment regulatory ability thereof.

### 6. In Vivo Anti-Tumor Efficacy Validation of TMER CAR-M

This study preliminarily explored the tumor-targeting capability of the TMER CAR-M in vivo. The anti-tumor effect of TMER CAR-M was verified in a mouse HER2⁺-4T1 tumor model. The establishment of the HER2⁺-4T1 tumor model, including the treatment, is as follows:
(1) Mouse breast cancer HER2⁺-4T1 cells were cultured in a 1640 medium (Hyclone) at 37° C. and 5% CO2 in an incubator;
(2) Female BALB/c mice aged 4-6 weeks were acclimated in a constant-temperature sterile ventilated mouse raising room for more than one week before the study;
(3) HER2+-4T1 cells were expanded. After the one-week acclimation, HER2⁺-4T1 cells were digested, washed with PBS twice or thrice, and resuspended in PBS at a density of 10⁷ cells/mL. 100 μL of the cell resuspension was subcutaneously injected at the right upper limb of the mice;
(4) UTD-M, Empty, CAR-like, First generation CAR-M, and TMER CAR-M cells were cultured in a high-glucose DMEM medium at 37° C. and 5% CO₂ in an incubator;
(5) After the HER2⁺-4T1 cells formed a tumor, the mice were randomized into groups of 4 mice. 100 μL of PBS or cell resuspension was injected via the tail vein. 3×10⁶ cells were injected for the UTD-M, Empty, CAR-like, First generation CAR-M, and TMER CAR-M groups, and a second dose was administered on day 5 after the first dose at 3×10⁶ cells per mouse. Thus, the mice were given a total of two doses.
(6) The length and width of the tumor were measured by an electronic vernier caliper. The weight of the mice was measured by an electronic balance. The tumor volume=(length×width²)/2. The measurement was conducted once every two days, and finally, the survival curve of the mice was plotted.

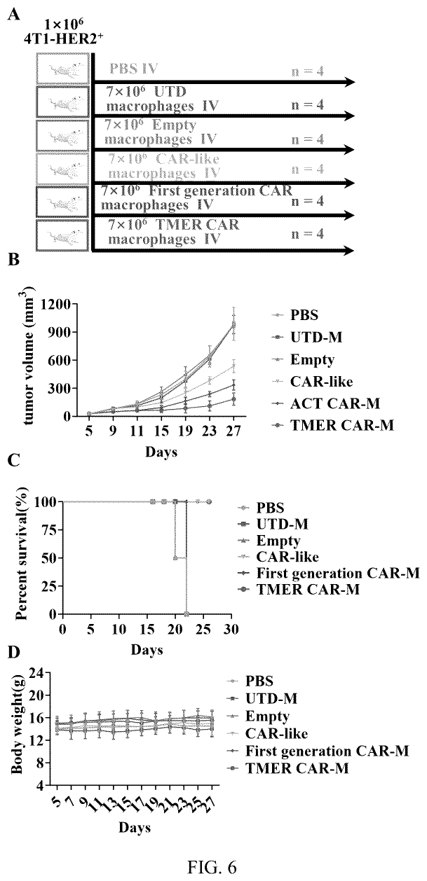

The results are shown in FIG. 6. FIG. 6A illustrates the treatment regimens; FIG. 6B shows the tumor volume of mice in different treatment groups and the control group during the treatment; FIG. 6C illustrates the survival of mice in different treatment groups and the control group; FIG. 6D illustrates the weight of mice in different groups. The study results show that compared with the CAR-like group and First generation CAR-M group, the tumor growth in the TMER CAR-M group was significantly inhibited, and the survival was prolonged, suggesting a significant therapeutic effect. In addition, compared with the CAR-like group and the First generation CAR-M group, the body weight of the TMER CAR-M group exhibited no significant changes, indicating good safety.

### 7. In Vivo Validation of Effect of TMER CAR-M in Treating Peritoneal Tumors

The mouse HER2⁺-4T1 tumor model confirmed the anti-tumor effect of the TMER CAR-M. The anti-tumor effect of the TMER CAR-M according to the present invention was further verified in a human ovarian cancer SK-OV-3 (high HER2 expression) peritoneal tumor model. The establishment of the SK-OV-3 tumor model, including the treatment, is as follows:
(1) Human ovarian cancer SK-OV-3 cells were cultured in a DMEM medium (Hyclone) at 37° C. and 5% CO₂ in an incubator;
(2) Nude mice aged 4-6 weeks were acclimated in a constant-temperature sterile ventilated mouse raising room for more than one week before the study;
(3) SK-OV-3 cells were expanded. After the one-week acclimation, SK-OV-3 cells were digested, washed with PBS twice or thrice, and resuspended in PBS at a density of 5×10⁶ cells/mL. 100 μL of the cell resuspension was intraperitoneally injected in mice;
(4) UTD-M and TMER CAR-M cells derived from human monocyte THP-1 were cultured in a 1640 medium at 37° C. and 5% CO₂ in an incubator containing;
(5) 2-4 h after SK-OV-3 cells were intraperitoneally injected, the mice were randomized into groups of 5 mice. 100 μL of PBS or cell resuspension was injected intraperitoneally. One dose of 1.5×10⁶ cells was injected for the UTD-M and TMER CAR-M groups.
(6) The tumor growth in the mice was monitored in real time using an IVIS Lumina II living animal imaging system.

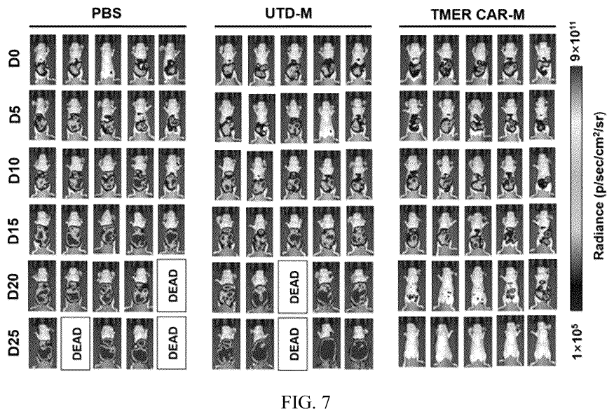

The results, as shown in FIG. 7, show that compared with the PBS group and UTD-M group, the fluorescence intensity in the TMER CAR-M group indicated a gradually reduced tumor cell count, and all the mice in the treatment groups survived during the study. The fluorescence intensities in the PBS group and UTD-M group gradually increased, and deaths were observed, suggesting that compared with the PBS group and UTD-M group, the SK-OV-3 tumors in the TMER CAR-M group were significantly inhibited, and the TMER CAR-M achieves a significant therapeutic effect. The TMER CAR-M can effectively inhibit the growth of SK-OV-3 tumors.

The above examples are only intended to illustrate the preferred embodiments of the present invention rather than limit the scope of the present invention. Various modifications and improvements made to the technical solutions of the present invention by those skilled in the art without departing from the spirit of the present invention should fall within the protection scope defined by the claims of the present invention.

What is claimed is:

1. A tumor microenvironment-regulatory CAR-monocyte/macrophage, wherein the CAR-monocyte/macrophage is a complex structure comprising a chimeric antigen receptor capable of producing GM-CSF expressed intracellularly and secreted extracellularly to promote the differentiation of the CAR-monocyte/macrophage to an M1 macrophage and further regulate the tumor microenvironment; the GM-CSF comprises the nucleotide sequence set forth in SEQ ID NO: 1 or SEQ ID NO: 9.

2. A chimeric antigen receptor, comprising GM-CSF, a HER2 scFv, a transmembrane region of α1β1 integrin, an intracellular region of α1β1 integrin, and an intracellular region of FcγRI transmembrane signaling domain.

3. The chimeric antigen receptor according to claim 2, wherein the nucleotide sequences of the GM-CSF, the HER2 scFv, the transmembrane region of α1β1 integrin, the intracellular region of α1β1 integrin, and the intracellular region of FcγRI transmembrane signaling domain are set forth in SEQ ID NO: 1/SEQ ID NO: 9, SEQ ID NO: 2, SEQ ID NO: 3/SEQ ID NO: 10, SEQ ID NO: 4/SEQ ID NO: 11, and SEQ ID NO: 5/SEQ ID NO: 12, respectively.

4. A recombinant vector, comprising the chimeric antigen receptor according to claim 2.

5. A method for preparing the tumor microenvironment-regulatory CAR-monocyte/macrophage, comprising: constructing the recombinant vector, and transfecting the CAR-monocyte/macrophage with the recombinant vector;
the recombinant vector comprising the chimeric antigen receptor according to claim 2;
the tumor microenvironment-regulatory CAR-monocyte/macrophage is a complex structure comprising a chimeric antigen receptor capable of producing GM-CSF expressed intracellularly and secreted extracellularly to promote the differentiation of the CAR-monocyte/macrophage to an M1 macrophage and further regulate the tumor microenvironment; the GM-CSF comprises the nucleotide sequence set forth in SEQ ID NO: 1 or SEQ ID NO: 9.

6. The method according to claim 5, wherein constructing the recombinant vector comprises:
(1) constructing the chimeric antigen receptor: connecting the HER2 scFv, the transmembrane region of α1β1 integrin, the intracellular region of α1β1 integrin, and the intracellular region of FcγRI transmembrane signaling domain via a linker, and connecting the GM-CSF via a P2A to synthesize the chimeric antigen receptor; and
(2) recombining the chimeric antigen receptor with a lentiviral vector to construct the recombinant vector.

7. A pharmaceutical composition, comprising: the CAR-monocyte/macrophage according to claim 1 and a pharmaceutically acceptable excipient or carrier.

8. Use of the CAR-monocyte/macrophage according to claim 1 in preparing a medicament for promoting the differentiation to an M1 macrophage.

9. Use of the CAR-monocyte/macrophage according to claim 1 in preparing a medicament for treating a solid tumor.

10. A recombinant vector, comprising the chimeric antigen receptor according to claim 3.

11. A method for preparing the tumor microenvironment-regulatory CAR-monocyte/macrophage, comprising: constructing the recombinant vector, and transfecting the CAR-monocyte/macrophage with the recombinant vector;
the recombinant vector comprising the chimeric antigen receptor according to claim 3;
the tumor microenvironment-regulatory CAR-monocyte/macrophage is a complex structure comprising a chimeric antigen receptor capable of producing GM-CSF expressed intracellularly and secreted extracellularly to promote the differentiation of the CAR-monocyte/macrophage to an M1 macrophage and further regulate the tumor microenvironment; the GM-CSF comprises the nucleotide sequence set forth in SEQ ID NO:
1 or SEQ ID NO: 9.

12. A pharmaceutical composition, comprising: the chimeric antigen receptor according to claim 2 and a pharmaceutically acceptable excipient or carrier.

13. A pharmaceutical composition, comprising: the recombinant vector and a pharmaceutically acceptable excipient or carrier;
the recombinant vector comprising the chimeric antigen receptor according to claim 2.

14. A pharmaceutical composition, comprising: the recombinant vector, and a pharmaceutically acceptable excipient or carrier;
the recombinant vector comprising the chimeric antigen receptor according to claim 3.
